(19) United States
(12) Patent Application Publication
(10) Pub. No.: US 2014/0193426 A1
(43) Pub. Date: Jul. 10, 2014

(54) MARKERS FOR EARLY DIAGNOSIS OF OVARIAN CANCER, MONITORING DURING THERAPY, AND NEW THERAPY OPTIONS DURING AND AFTER CHEMOTHERAPY

(71) Applicant: The Board of Trustees of the University of Arkansas, Little Rock, AR (US)

(72) Inventors: Timothy J. O'Brien, Little Rock, AR (US); John Beard, Maumelle, AR (US); Wilbur C. Hitt, JR., Little Rock, AR (US)

(73) Assignee: The Board of Trustees of the University of Arkansas, Little Rock, AR (US)

(21) Appl. No.: 14/107,751

(22) Filed: Dec. 16, 2013

Related U.S. Application Data
(63) Continuation of application No. PCT/US2012/043027, filed on Jun. 18, 2012.
(60) Provisional application No. 61/573,759, filed on Sep. 12, 2011.
(60) Provisional application No. 61/520,958, filed on Jun. 17, 2011.

(51) Int. Cl. C12Q 1/37

(52) U.S. Cl. CPC C12Q 1/37
USPC 424/158.1; 435/23

(57) ABSTRACT

The inventors have identified several proteases and a protease inhibitor that are overexpressed in ovarian cancer tumors. They have developed monoclonal antibodies against the proteins and shown that they can be detected in serum and the levels of the proteins in serum fluctuate during cancer treatment. They have shown that serum assays for the proteases and protease inhibitor can be used for early detection of ovarian cancer, and for monitoring cancer treatment.

### BACKGROUND

Ovarian cancer remains the number one killer of women with gynecologic disease. More than 15,000 new cases are recognized each year in the United States with 25,000 women dying of their disease on an annual basis. Two major challenges remain to be addressed to provide reasonable possibilities for better outcomes for women with ovarian cancer. Moving diagnosis from stage III-IV to stage I-II can have a major impact because currently 75% of women are diagnosed with stage III-IV disease when the five year survival is approximately 25%. Recognizing early stage disease (stage I-II) would elevate survival statistics to 85% for these women. Concomitant with late stage disease is the related problem of recurrence in these women after surgery and chemotherapy. Currently no real alternate therapies are available after disease becomes resistant to current chemotherapy regimens. These factors dictate therefore that additional markers should have a capacity for early diagnosis complimenting CA125 and for identifying new targets for therapy in women diagnosed with late stage disease.

### SUMMARY

The inventors have discovered by PCR amplification of RNA from tumor samples that several proteases and a protease inhibitor (ALP) are often elevated in tumor samples, including ovarian cancer, prostate cancer, and cervical cancer samples. The elevated proteases include Stratum Corneum Chymotryptic Enzyme (SCCE), more recently renamed as KLK7; Hepsin; and several new sequences which we described as the TADG series or the

Tumor Associated Diagnostic Gene series which included TADG-14, later renamed KLK8; TADG-15 also named Matriptase; TADG-12 and variants now renamed as TMPRSS3; the metalloprotease family member MMP-7 (Pump1), and kallikrein-related peptidase 6 (KLK6, also known as human kallikrein 6 or hK6). The serine protease inhibitor ALP (Antiluekoprotinase or SLPI), was also found to be elevated in many cancer patients.

The inventors have produced polyclonal and monoclonal antibodies against these proteins and used the antibodies to show by immunohistochemical staining that the proteins are found in tumor samples. They have also used these newly developed antibodies to develop serum assays for the proteins and have shown that the serum levels of the proteins are often elevated in cancer patients, including early stage ovarian cancer patients.

The inventors have found that a combination of the serum levels of proteins selected from these proteases listed above, the protease inhibitor ALP, and the well known cancer marker CA125, can be used for early detection of cancer, including ovarian cancer.

Thus, one embodiment of the invention provides a method of early diagnosis of cancer comprising: (a) measuring in a human blood sample (e.g., whole blood, serum, or plasma) protein levels of two or more proteins selected from the group consisting of CA125, TADG14, TADG15, TADG12, SCCE, MMP-7, ALP, KLK6, and hepsin; and (b) comparing the levels of the two or more proteins to normal range levels of the two or more proteins to identify whether the level of at least one of the two or more proteins is elevated. If at least one of the levels of the two or more proteins is elevated, the human may have cancer.

The method typically further involves conducting one or more further tests on the human to identify if the human has cancer.

Another embodiment provides a method of monitoring progress of cancer in a cancer patient comprising: (a) measuring in the cancer patient blood (e.g., whole blood, serum, or plasma) protein levels of two or more proteins selected from the group consisting of CA125, TADG14, TADG15, TADG12, SCCE, MMP-7, ALP, KLK6, and hepsin; and (b) comparing the levels of the two or more proteins to normal range levels of the two or more proteins and/or to previous levels of the two or more proteins in the same patient to identify whether the level of at least one of the two or more proteins is elevated above normal range levels or is increasing or decreasing.

Another embodiment provides a method of treating cancer comprising administering a protease inhibitor. In a more specific embodiment, the protease inhibitor is Bowman-Birk inhibitor, ALP, aprotinin, HAI-1, PEBP (phosphatidylethanoloamine-binding protein), FOY-305 (FOYPAN), probucol, or an antibody or antibodies against one or more of the proteases selected from the group consisting of: TADG12, TADG14, TADG15, SCC, MMP-7, KLK6, and hepsin.

Bowman-Birk inhibitor inhibits TADG15 (matriptase), and possibly other proteases. ALP inhibits SCCE (KLK-7), and possibly other proteases. HAI-1 inhibits TADG15 (matripatase), and possibly other proteases. FOY-305 inhibits TADG15 (matriptase). Probucol inhibits hepsin (Chevillet J R, et al., Identification and characterization of small-molecule inhibitors of hepsin. Mol. Cancer Ther. 7(10): 3343-3351, 2008).

The Bowman-Birk inhibitor is reviewed in Birk, Y. (1985), The Bowman-Birk inhibitor. Trypsin- and chymotrypsin-inhibitor from soybeans. International Journal of Peptide and Protein Research, 25: 113-131.

In other embodiments, the protease inhibitor is a cyclic peptide 20 amino acids or less having the structure:

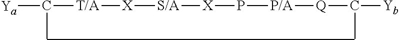

where Yₐ and Y_{b} are each optionally present, and if present are a peptide of 1-11 amino acid residues, and where Yₐ and Y_{b} collectively comprise 0-11 amino acid residues. In this structure the single-letter amino acid abbreviations are used; a slashed pair of letters indicate either of the amino acids designated by the letters can be used at that position; and X is any amino acid. The two cysteine residues are linked together by a disulfide bond, shown by the solid line, to cyclize the peptide. In one embodiment, the peptide is a cyclic 9-mer with the sequence CTKSNPPQC (SEQ ID NO:1), optionally with Yₐ and Y_{b} peptides at the N and C termini. In another embodiment, the peptide is a cyclic 9-mer with the sequence CALSYPAQC (SEQ ID NO:2), optionally with Yₐ and Y_{b} peptides at the N and C termini. (McBride et al. Synthetic peptide mimics of the Bowman-Birk inhibitor protein, Curr. Med. Chem. 2001, 8:909-917.) In particular embodiments, Yₐ and Y_{b} are each 1 amino acid residue. In particular embodiments Yₐ and Y_{b} are each absent.

Other conventional known protease inhibitors can also be used. For instance, leupeptin inhibits many serine proteases, probably including some of the group consisting of TADG12, TADG14, TADG15, SCC, MMP-7, KLK6, and hepsin.

Another embodiment provides a method of treating cancer comprising: measuring in a cancer patient blood protein levels of one or more proteases selected from the group consisting of TADG12, TADG14, TADG15, SCCE, MMP-7, KLK6, and hepsin; comparing the levels of the one or more proteases to normal range levels of the one or more proteases and/or to previous levels of the one or more proteases in the same patient to identify whether the level of at least one of the one or more proteases is elevated above normal range levels or is increasing; and if the level of at least one of the one or more proteases is elevated or increasing, treating the patient with an inhibitor of the elevated or increasing at least one protease.

### BRIEF DESCRIPTION OF THE DRAWINGS

FIG. 1. DNA electrophoresis showing PCR amplification of antisense 1 (AS1) and antisense 2 (AS2) segments of consensus active site segments of serine protease genes, amplified with redundant primers, in tumor samples and not in normal ovary tissue.

FIG. 2. Alignment of TADG-12 protein and two of its variants. Portions of the variants that are nonhomolgous to TADG-12 are underlined.

FIG. 3. TADG-15 PCR on normal ovary, Stage 1 ovarian cancer, and Stage III ovarian cancer samples.

FIG. 4. Hepsin and Hesin V variant PCR on prostate tumor cDNA.

FIG. 5. Relative expression of TADG-15 in normal, benign, and ovarian tumor tissues by Real-time PCR. Tumors are classified by cancer stage and histopathology. Means and standard deviation are shown.

FIG. 6. Relative expression of TADG-14 in normal, benign, and ovarian tumor tissues by Real-time PCR. Tumors are classified by cancer stage and histopathology. Means and standard deviation are shown.

FIG. 7. Relative expression of Hepsin in normal, benign, and ovarian tumor tissues by Real-time PCR. Tumors are classified by cancer stage and histopathology. Means and standard deviation are shown.

FIG. 8. Relative expression of TADG-12D in normal, benign, and ovarian tumor tissues by Real-time PCR. Tumors are classified by cancer stage and histopathology. Means and standard deviation are shown.

FIG. 9. Relative expression of ALP in normal, benign, and ovarian tumor tissues by Real-time PCR. Tumors are classified by cancer stage and histopathology. Means and standard deviation are shown.

FIG. 10. Relative expression of SCCE and MMP-7 in normal, benign, and ovarian tumor tissues by Real-time PCR. Tumors are classified by cancer stage and histopathology. Means and individual data are shown.

FIG. 11. SDS-PAGE showing purified protein example TADG-15.

FIG. 12. ELISA assay standard curve for TADG-15 (Matriptase).

FIG. 13A-F. Serum protein levels of markers by sandwich ELISA assay for candidate marker proteins in Patient 1, a Stage IV ovarian cancer patient. (TADG-14 and TADG-15 are referred to as T-14 and T-15).

FIG. 14A-B. Serum protein levels of markers by sandwich ELISA assay for candidate marker proteins in Patient 2, a Stage III ovarian cancer patient. Patient 2 demonstrates the presence of TADG-15 early in the therapeutic regimen of this patient with its reactivation in mid-therapy. Also patient 2 elaborates both TADG-14 and MMP-7 in a coordinated fashion during therapy.

FIG. 15. Serum protein levels of marker proteins by sandwich ELISA assay in Patient 3, a Stage IV ovarian cancer patient.

FIG. 16. Serum protein levels of marker proteins by sandwich ELISA assay in Patient 4, a Stage I ovarian cancer patient.

FIG. 17. Serum protein levels of marker proteins by sandwich ELISA assay in Cervical Cancer Patient 1. Panel A shows overexpression of ALP and panel B shows overexpression of TADG-15.

FIG. 18. Serum protein levels of marker proteins by sandwich ELISA assay in Cervical Cancer Patient 2, showing overexpression of SCCE, TADG-15, and CA125.

FIG. 19A-B. Serum protein levels of marker proteins by sandwich ELISA assay in Cervical Cancer Patient 3, showing overexpression of Hepsin and ALP. Panel B is the same information as panel A with the scale up to 250 ng/L in panel B and 800 ng/L in panel A, and the ALP data shown only in panel A.

FIG. 20. Serum protein levels of marker proteins by sandwich ELISA assay in Cervical Cancer Patient 4, showing overexpression of TADG-15 and ALP.

FIG. 21. Serum protein levels of marker proteins by sandwich ELISA assay in Cervical Cancer Patient 5, showing overexpression of Hepsin, TADG-15, SCCE, and ALP.

FIG. 22. Serum protein levels of marker proteins by sandwich ELISA assay in Cervical Cancer Patient 6, showing overexpression of TADG-15.

FIG. 23. Marker expression and potential for monitoring, diagnostics, and targeted therapy in other cancers, based on real-time PCR of tumor samples.

### DETAILED DESCRIPTION

In an effort to identify markers that can provide early diagnosis of ovarian cancer or new targets for therapy of women diagnosed with late stage ovarian cancer, we have put in place a set of criteria for identifying new markers which might be considered for both roles, viz. complementing CA125 in early diagnosis and identifying new targets for therapy in women diagnosed with late stage disease.

TABLE 1
Criteria for new markers for diagnosis, monitoring, and identifying
targets for therapeutic intervention.

Highly overexpressed in tumor cells
Secreted or released from tumor cells
Relatively low molecular weight (<50,000)
Low or no expression in normal adult tissue
Reasonable half-life in serum/bodily fluids
Expressed and secreted in early stage disease
Involved with tumor growth and/or spread

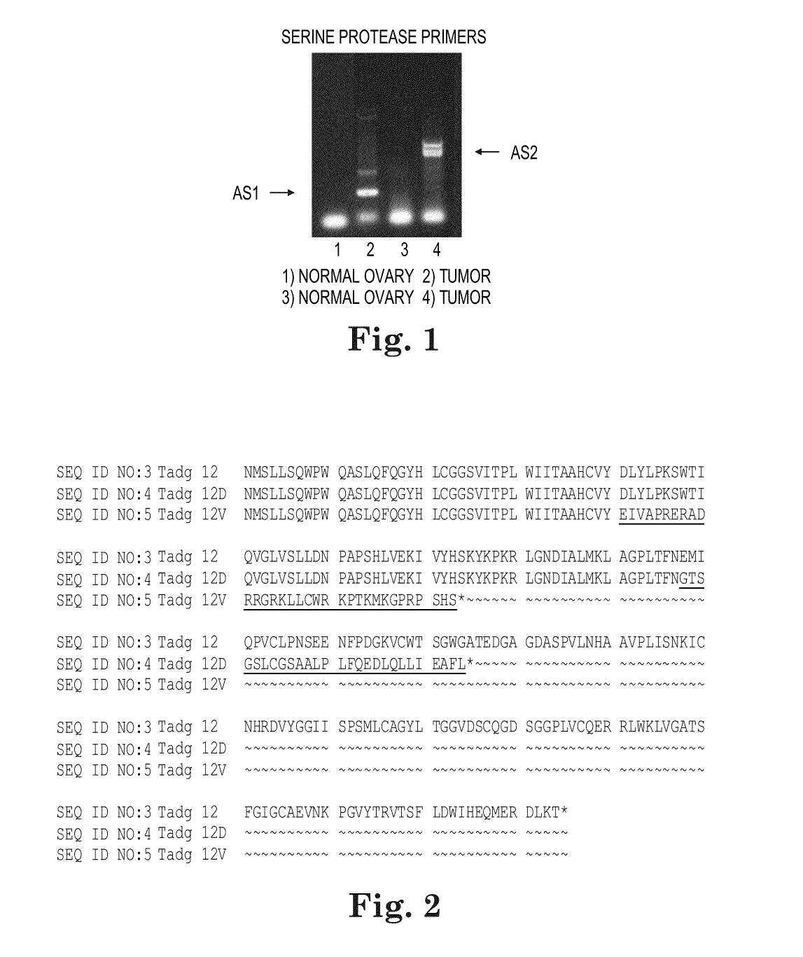

To address the challenges of identifying such markers we developed a strategy of searching for families of genes which might be involved in tumor growth and spread and which fulfilled the other criteria by synthesizing redundant primers to signature sequences of multiple families of genes, e.g. the serine protease family. Two examples of gene segments amplified in this way are the antisense 1 (AS1) and antisense 2 (AS2) segments amplified with redundant primer to the coding portions for segments of the active site in serine proteases. These were amplified from ovarian tumor tissue and were not detectable in normal ovary tissue (FIG. 1).

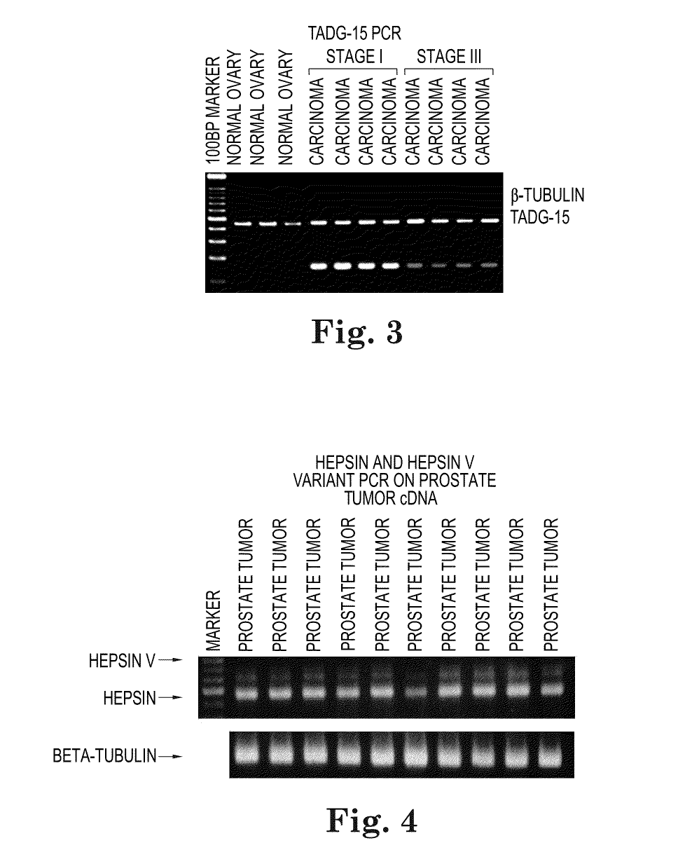

With this approach it is possible to PCR amplify all family members of a given family and to determine their value as ovarian cancer markers by comparing amplified products from carcinoma tissues versus normal ovarian epithelium. Such was the case when we compared the expression of the serine protease family in normal and carcinoma tissue. In this case we then sub-cloned the amplified PCR products and sequenced the sub-clones to identify the genes which were amplified. To our surprise we identified several proteases which heretofore were not associated with carcinoma but may have functional activity in tumor growth and spread. An example of such a gene was the Stratum Corneum Chymotriptic Enzyme (SCCE), more recently renamed as KLK7, which was primarily associated with skin cell desquamation and which was originally cloned by Egelrud (Hansson et al., 1994) and demonstrated to be a primary factor in skin desquamation. Also indentified in this process of sub-cloning and sequencing were the genes Hepsin, originally cloned from hepatocytes, and several new sequences which we described as the TADG series or the Tumor Associated Diagnostic Gene series which included TADG-14, later renamed KLK8, TADG-15 also named Matriptase, TADG-12 and variants now renamed as TMPRSS3. Using other family signature sequences to look for other candidates, we also identified the metalloprotease family member MMP-7 (Pump1) and the serine protease inhibitor ALP (Antiluekoprotinase or SLPI). Initial validation of these genes and their expression in ovarian cancer was carried out by PCR of a panel of tumors and by Northern blot analysis to demonstrate the presence of RNA in tumors of ovarian origin. Examples of such validation are presented below for TADG-15 (Matriptase) (FIG. 3) and Hepsin in prostate cancer (FIG. 4).

The term “TADG-12” as used herein includes variants of TADG-12, particularly TADG-12D and TADG-12V. The sequences of TADG-12 (SEQ ID NO:3), TADG-12D (SEQ ID NO:4), and TADG-12V (SEQ ID NO:5) are shown in FIG. 2.

Further validation was provided by immunohistochemistry utilizing polyclonal antibodies produced in rabbits by immunizing them with 12 mer peptides from each gene product coupled to a polylysine backbone. (Immunohistochemical stain data not shown.)

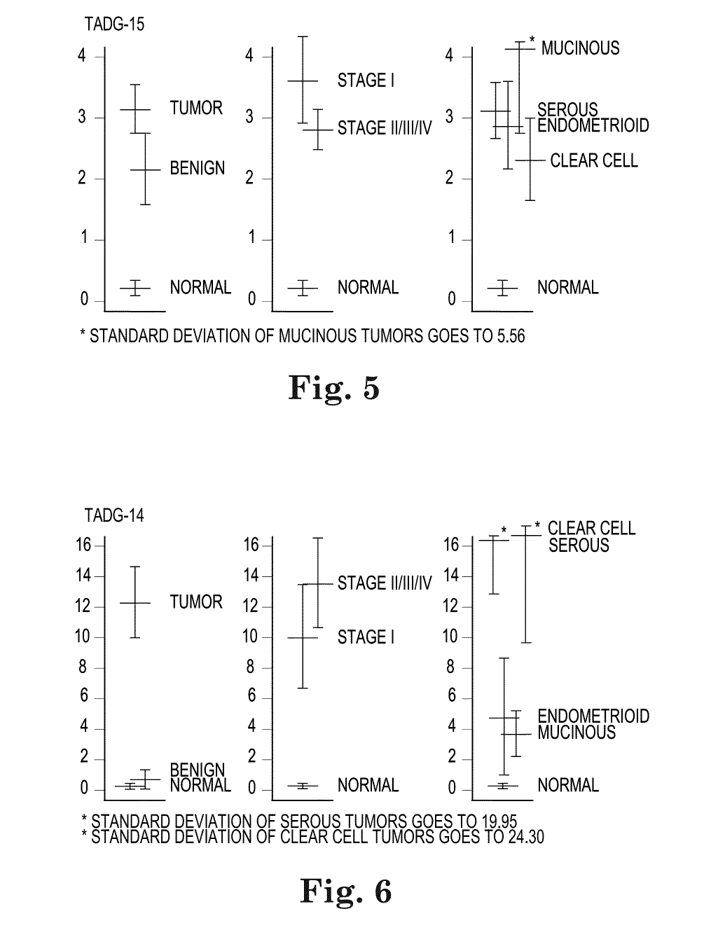

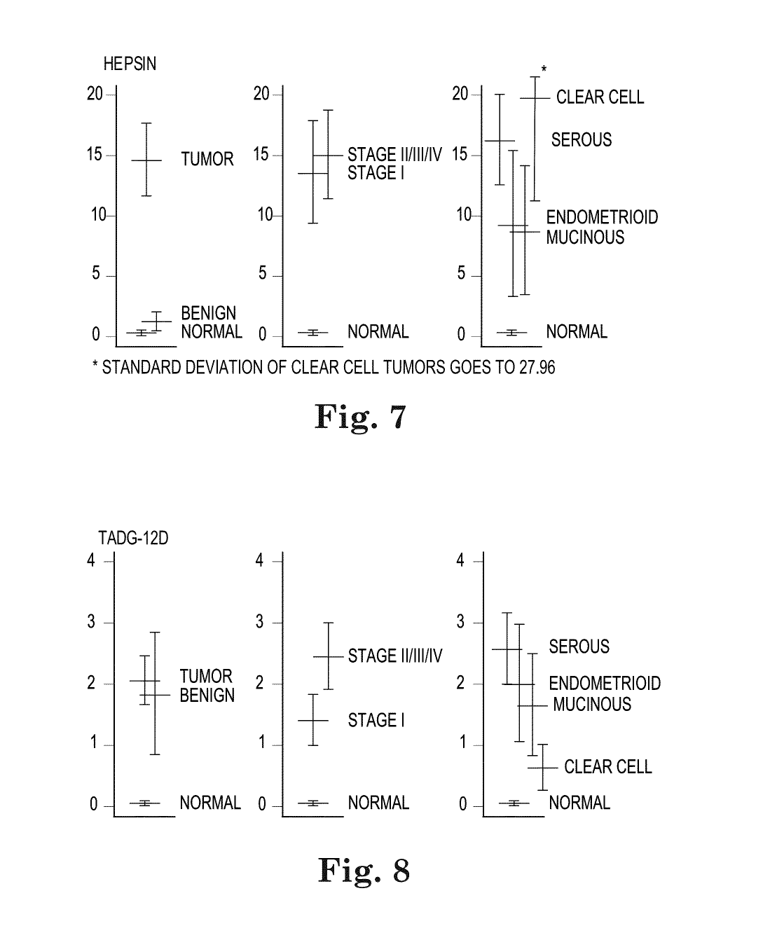

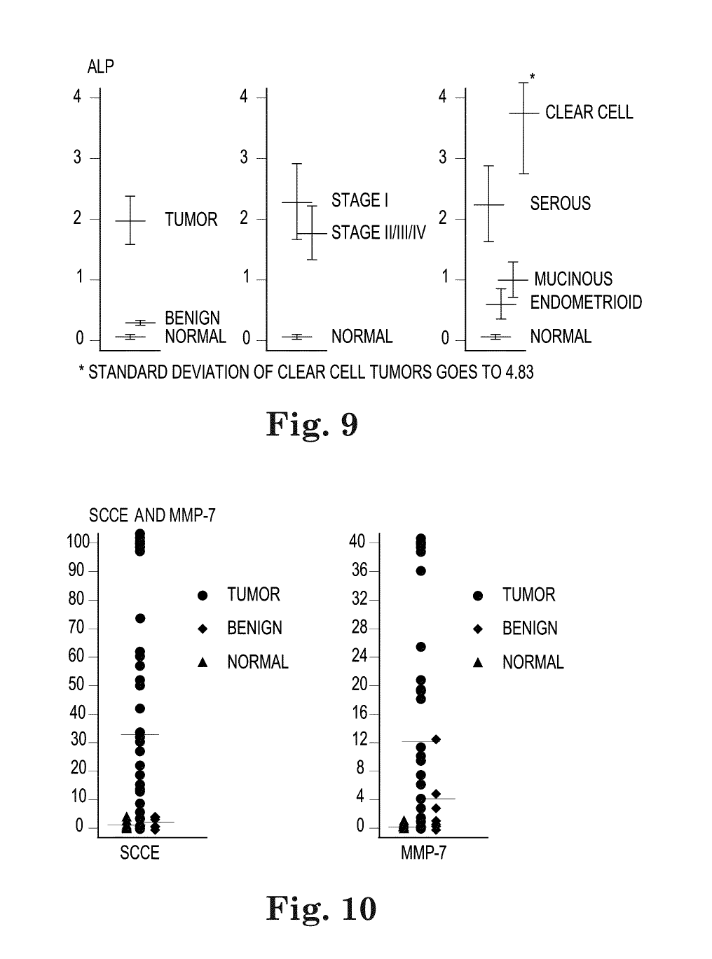

Further validation of overexpression of these genes in ovarian cancer was obtained using Real-Time PCR to quantify the transcript number in individual tumors from a panel of 55 tumors which included early stage, late stage, and many sub-groups of ovarian cancer including mucinous, serous, clear cell, and endometrioid types. These data are presented in FIGS. 5-10 for each gene product as represented in normal, benign, and carcinoma, in early and late stages, and in the sub-groups of cancer types described above.

In FIGS. 5-10 we observe the clear cut overexpression of the selected gene products in ovarian carcinomas including TADG-14 (KLK8), TADG-15 (Matriptase), SCCE (KLK7), TADG-12 (TMPRSS3), MMP-7 (Pump-1), Hepsin (TMPRSS1) and ALP (SLPI). The data is real-time PCR from tumor tissue samples. Furthermore, we observe overexpression of individual markers in specific subgroups of tumors. For instance, Hepsin is overexpressed in all subgroups tested whereas TADG-14 (KLK8) shows high overexpression for the clear cell and serous sub-types but more moderate overexpression for the mucinous and endometrioid sub-types.

Similarly ALP is highly elevated in clear cell and mucinous sub-types while only moderately elevated in endometrioid and serous sub-types. Also of note is the observation that some markers will distinguish benign disease from malignant disease more effectively than others, e.g. TADG-14 (KLK8), Hepsin (TMPRSS1), ALP (SLPI), SCCE (KLK7), and MMP-7 all discriminate malignant from benign disease and as such can be an effective panel for early detection of ovarian cancer.

In assessing the expression of these genes in individual tumors we can determine the overexpression of markers in early stage (I-II) disease to evaluate the potential usefulness of this panel in detecting early stage disease. In a group of 53 tumors, 25 early stage (I-II) and 28 late stage (III-IV), we found the following data as shown in Table 2. The data is real-time PCR of tumor samples.

TABLE 2
Tumor recognition by the markers CA125, TADG-15,
Hepsin, TADG-14 and TADG-12D. The method is real-
time PCR of tumor samples. The number of tumors
recognized per total number of cases is shown.

| | | | | TUMOR HISTOLOGY | | |
|---|---|---|---|---|---|---|
| TUMOR STAGE | | | | | Endome- | Clear |
| Marker | I & II | III & IV | Serous | Mucinous | trioid | Cell |
| CA125 | 10/25 | 16/28 | 21/28 | 1/10 | 2/8 | 2/7 |
| TADG-14 | 13/25 | 20/28 | 21/28 | 4/10 | 3/8 | 5/7 |
| Hepsin | 20/25 | 20/28 | 24/28 | 6/10 | 3/8 | 7/7 |
| TADG-12D | 20/25 | 25/28 | 27/28 | 8/10 | 6/8 | 4/7 |
| TADG-15 | 13/25 | 16/28 | 15/28 | 6/10 | 5/8 | 3/7 |
| TOTAL | 25/25 | 28/28 | 28/28 | 10/10 | 8/8 | 7/7 |

Here when CA125 is included in our panel we find a panel which includes 53 out of 53 to be effective in recognizing all the early stage tumors as well as the late stage group and the individual sub-types. Most notably the complete group of mucinous tumors is recognized by this panel. These encouraging results stimulated interest in developing serum assays to determine if protein products of these genes fulfilled the criteria we set forth at the beginning of the study. Initially we moved forward by introducing the appropriate gene sequence into bacterial expression systems (E. coli) and had success in isolating and purifying protein products of these genes. However, we discovered that often proteins had to be denatured for isolation and purification followed by renaturation for use in assays or for developing antibodies. Such a process gave rise to relatively unstable configurations in the protein products and forced us to reconfigure our protein expression system. We converted all production to an insect cell line with the appropriate expression and secretion sequences to obtain product in serum free media as briefly described as follows.

Insect expression and purification—The following is an example of insect expression and purification of one of our recombinant proteins. The protease domain of the Matriptase gene was sent to Expression Systems, LLC (Woodland, Calif.) for transfer into their baculovirus expression system. The gene was amplified from the T-Vector clone and ligated into the pBacPAK8 HMB-His-TEV expression vector using Fse and Xba restriction sites. Positive clones were identified using colony PCR. The expression vector containing the gene was co-transfected with BestBac delta vCath/chiA (Expression Systems). After two rounds of virus amplification the titer was 5.2E8. T. ni Pro cells (Expression Systems) were then infected with the virus at a MOI of 1 and expression checked at 24 hours post infection.

We received positive virus from Expression Systems and set up production and purification in our laboratory. Starting material for purification was 300 mL of supernatant from a T. ni PRO infection at an MOI of 1.0. The supernatant was adjusted to 400 ml in 50 mM Phosphate pH 8.0, 300 mM NaCl, 10 mM Imidazole, and 0.05% Tween 20 at 5× concentration. The adjusted supernatant was mixed with 4 mL Ni-NTA beads (Qiagen) equilibrated with the buffer above for one hour. The beads were then washed twice with 250 mL of 50 mM Phosphate pH 8.0, 300 mM NaCl, 20 mM Imidazole, 0.05% Tween 20. The protein was eluted from the beads and collected in 1 mL fractions with 50 mM Phosphate pH 8.0, 300 mM NaCl, 250 mM Imidazole, 0.05% Tween 20, and 1 mM protease inhibitor 4-(2-Amino-ethly)benzenesulfonyl fluoride hydrochloride (AEBSF, Sigma). Fractions 5 through 8 were pooled and dialyzed in 4 L 50 mM Tris, 10% glycerol with two changes. AEBSF was added to the pooled fractions. Final yield was 3 mg at a concentration of 176 ug/ml. Our other recombinant proteins were also produced and purified in a similar fashion with comparable yields.

Monoclonal antibody production—The following is an example of monoclonal antibody production for one of our recombinant proteins. Purified recombinant Matriptase protease protein was sent to ProMab Biotechnologies, Inc. (Richmond, Calif.) for monoclonal antibody development. We received 10 clone supernatants which we screened against purified recombinant matriptase protein as well as other recombinant serine protease to test for specificity. We chose 7 clones and ProMab proceeded with final sub-cloning. ProMab performed ascites production for the seven clones for antibody production and then purified the antibodies using Protein G columns (GE Healthcare). We received purified monoclonal antibody in PBS buffer for the 7 clones from ProMab at concentrations ranging from 2.5 mg/ml to 7.5 mg/ml.

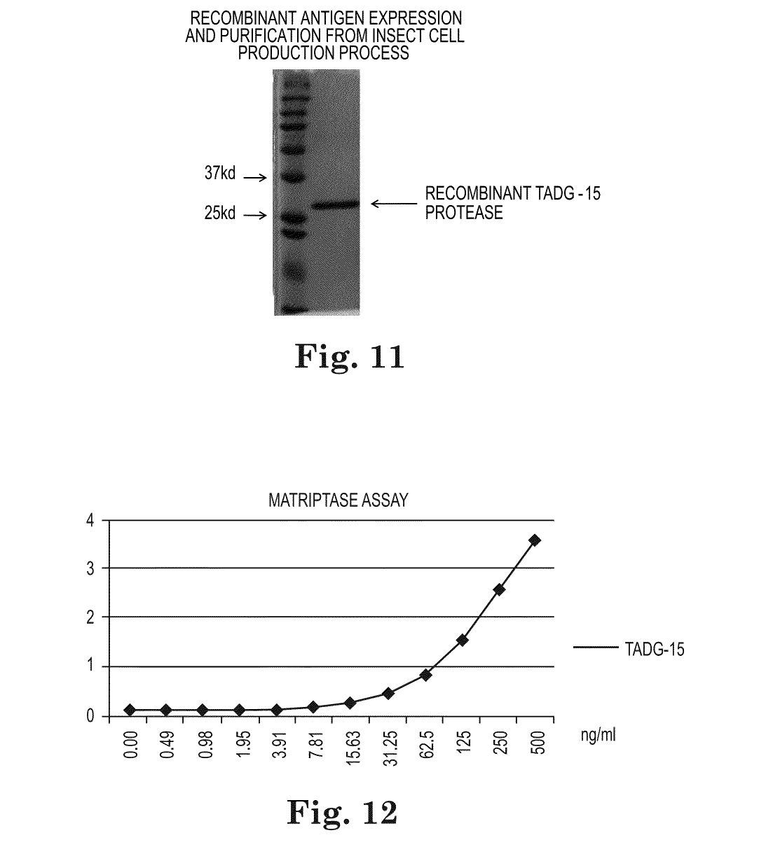

In most cases the insect system was productive in producing and secreting recombinant protein products for TADG-14, ALP, TADG-15, SCCE, and MMP-7. However for some gene products, especially the ones which were transmembrane enzymes, production often succeeded but secretion failed even though the transmembrane domains were not part of the recombinant proteins. In such cases, e.g. Hepsin and TADG-12D, cell lysates were used to purify recombinant proteins for antibody production in mice. As shown here, several proteins were purified to near 100% purity by standard columns for his-tag proteins (FIG. 11).

After immunization in mice followed by hybridoma production using the mouse polycytoma cell line and cloning of antibody producing hybridoma cell lines, multiple cell lines (8-10) were selected for antibody production via mouse ascites and purification of Protein G columns.

Pairs of antibodies, trapping and reporting, were selected by evaluation of all combinations of antibodies utilizing biotin labeling with streptavidin coupled HRP to report for each assay pair. Assay pairs were selected based on the most sensitive ELISA using recombinant antigen to develop standard curves. Assays were further examined for their capacity to recognize native antigen in test sera and were also evaluated for any cross-reactivity for other proteins in the group. A representative standard curve for Matriptase (TADG-15) is shown in FIG. 12.

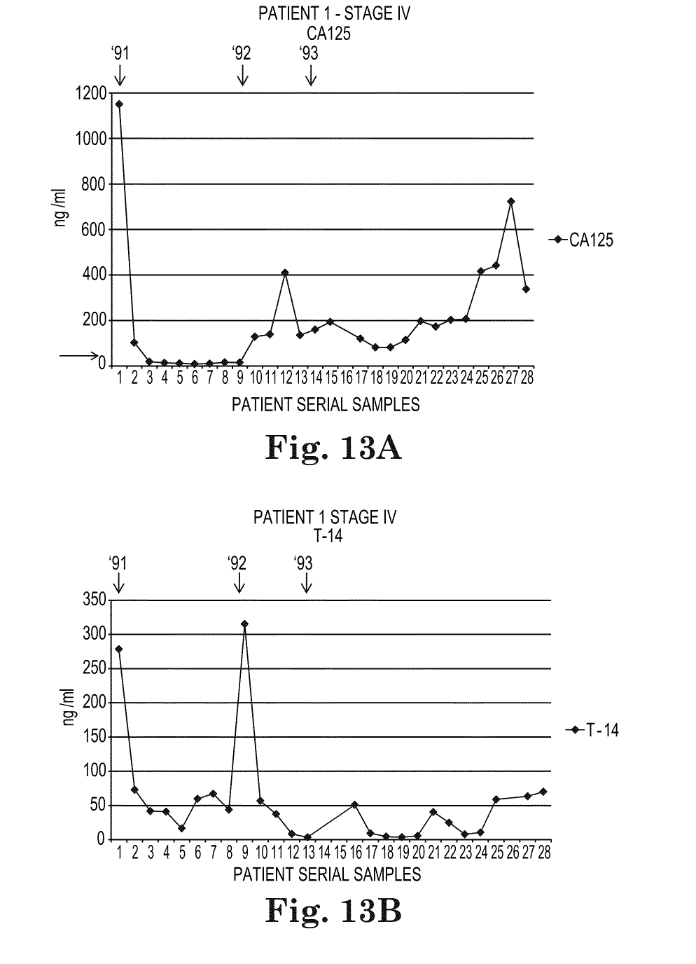

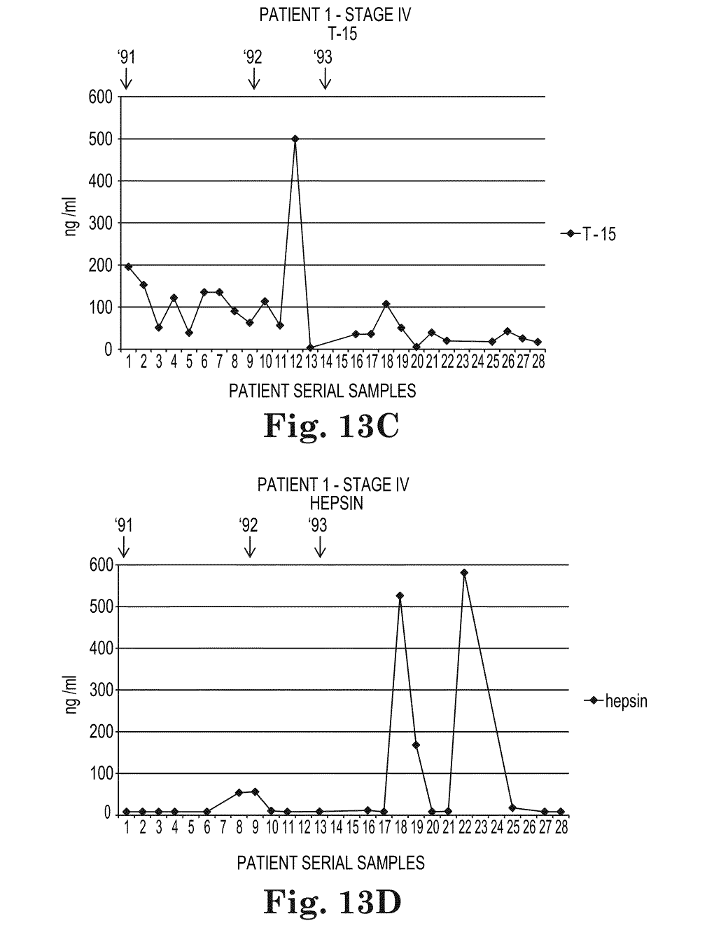

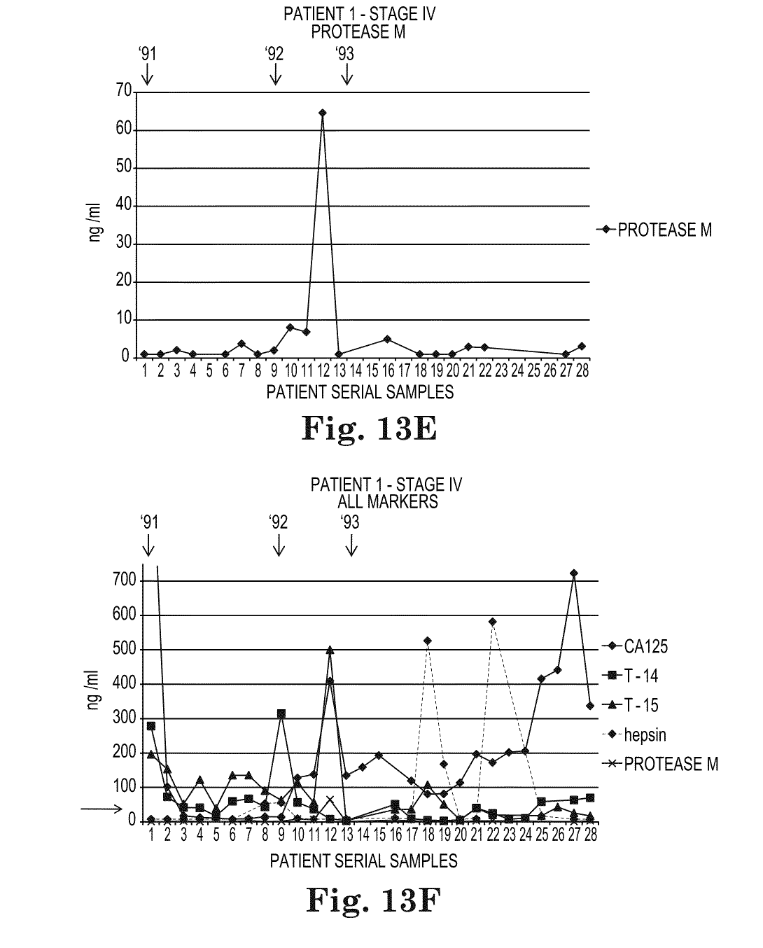

Evaluation of new markers in patients monitored for ongoing disease or recurrent disease was carried out on patients with low or no CA125 and also on patients who elaborated CA125. Examples of such patients are shown in FIGS. 13-16 where it can be noted that after surgery all markers tend to go toward background (see FIG. 13a, showing the drop from serial sample 1 before surgery to serial sample 2 after surgery).

Also noticeable was the fact that some markers spiked during chemotherapy which would indicate ongoing tumor growth and spread. Also noticeable in several patients is the fact that coordinated spiking and recession of markers would indicate a cascade of protease activity which can provide new indicators of active disease even when CA125 is quiescent or absent. Such could be the importance of these new markers which we have shown are over-expressed and exported directly from tumor cells thereby providing opportunities for new therapeutic intervention to mitigate tumor growth and spread.

FIG. 13 shows serum protein levels of markers by sandwich ELISA assay for candidate marker proteins in a Stage IV ovarian cancer patient. The data for this patient illustrate that individual markers are clearly expressed at specific intervals during chemotherapy treatment. Each marker may have its own specific profile (e.g. TADG-14 and Hepsin) or there may be coincidental expression of several markers at a specific time during therapy (e.g. TADG-15 and CA125).

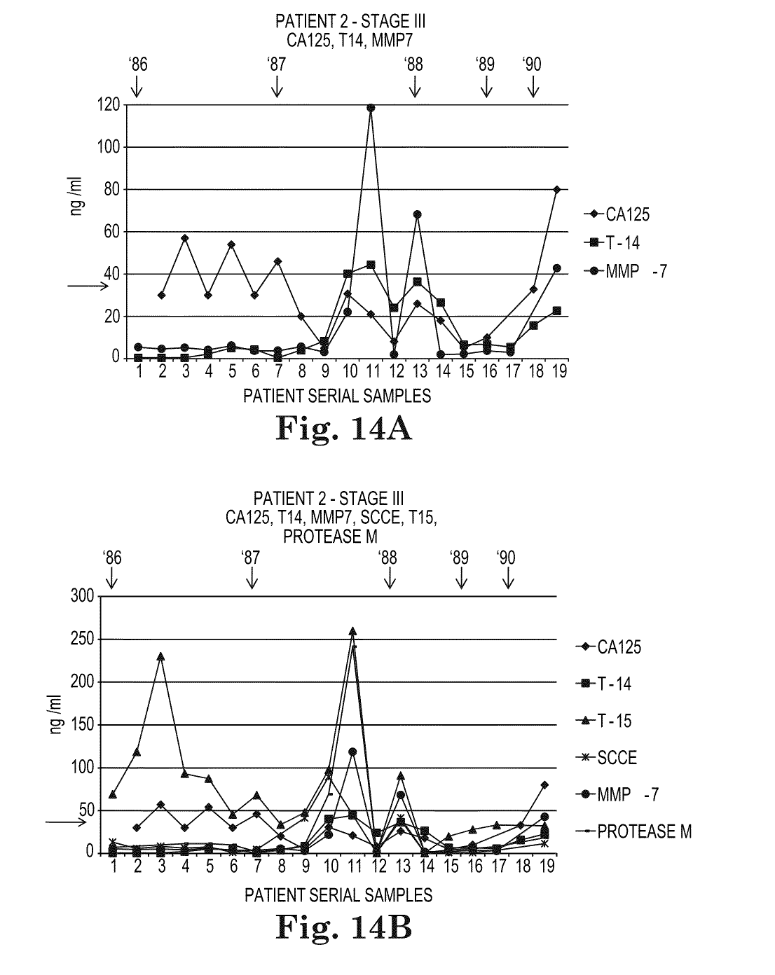

FIG. 14 shows the serum protein levels in Patient 2, a Stage III ovarian cancer patient. This patient demonstrates the presence of TADG-15 early in the therapeutic regimen, and its reactivation in mid-therapy. Also, patient 2 elaborates both TADG-14 and MMP-7 in a coordinated fashion during therapy.

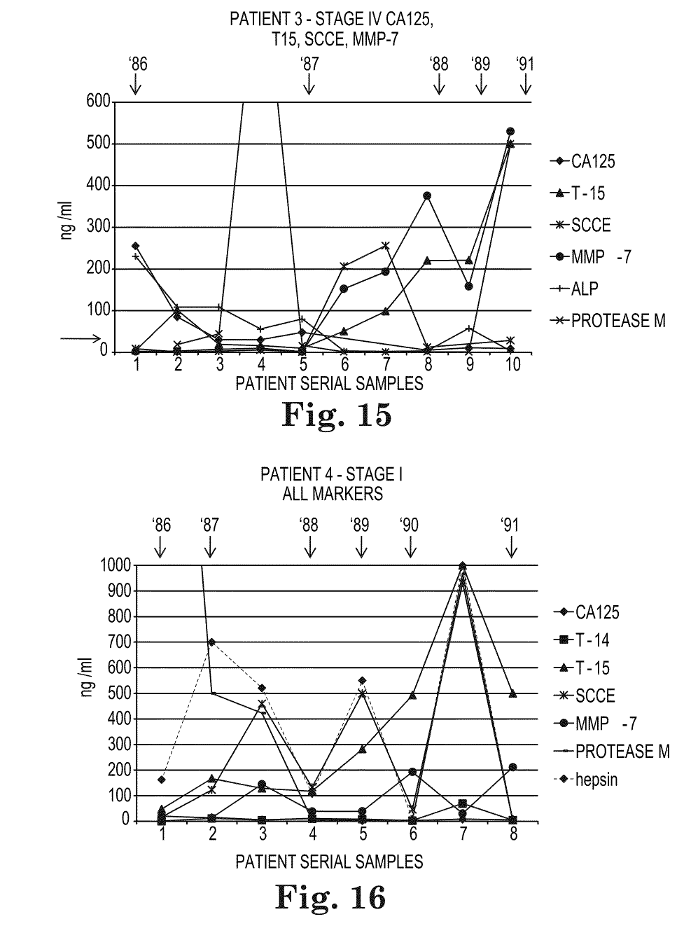

FIG. 15 shows the serum protein levels in Patient 3, a Stage IV ovarian cancer patient. This indicates the presence of TADG-15, SCCE, MMP-7, and Protease M during therapy.

FIG. 16 shows serum protein levels by sandwich ELISA assay in Patient 4, a Stage I ovarian cancer patient. These data show early expression of Hepsin with coordinated expression of Hepsin, TADG-15, and SCCE.

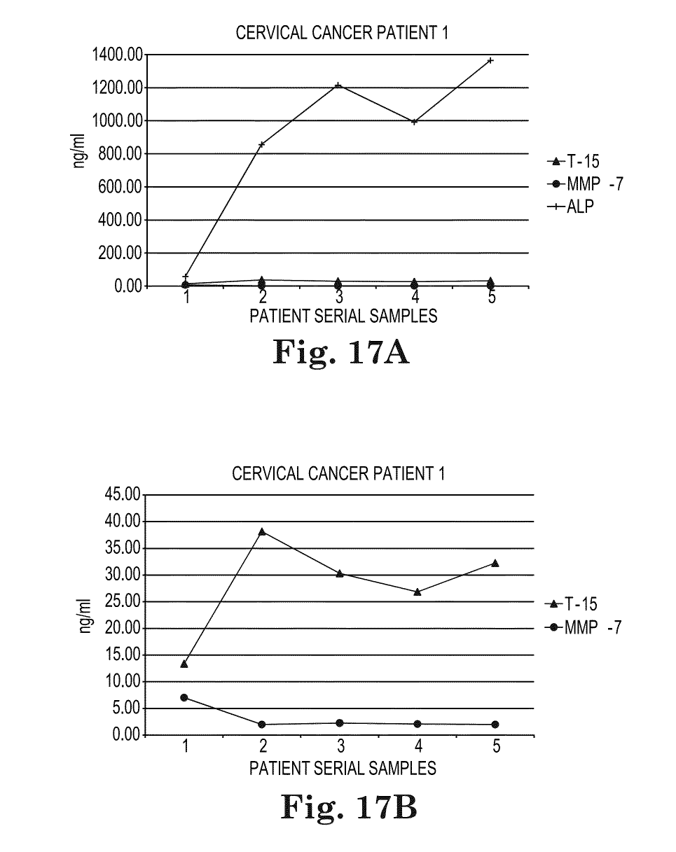

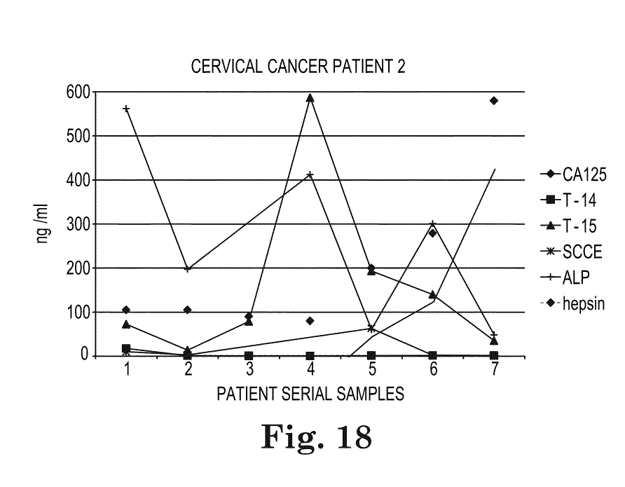

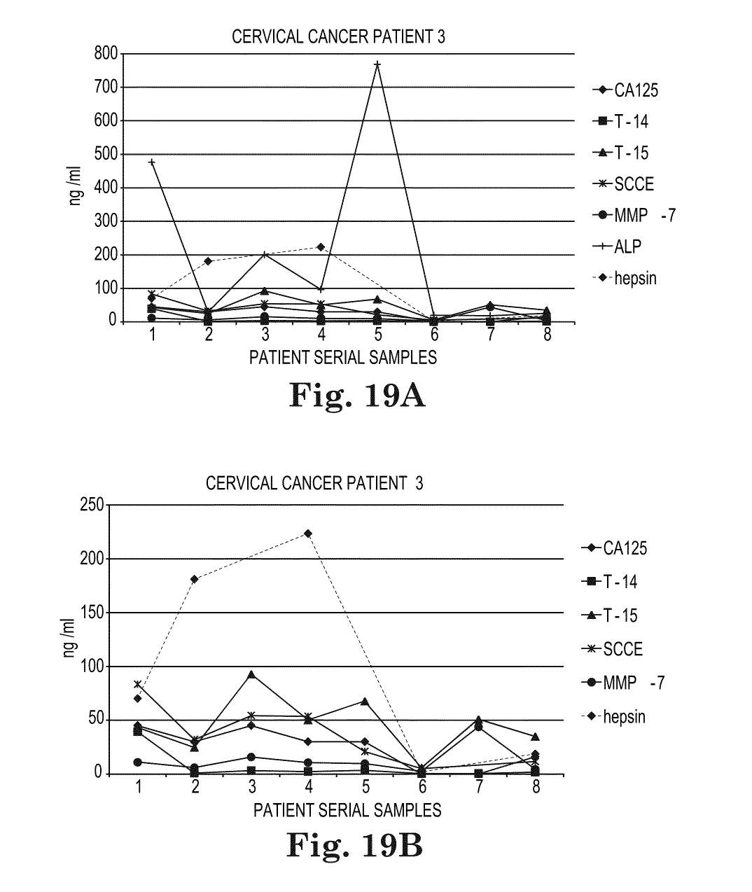

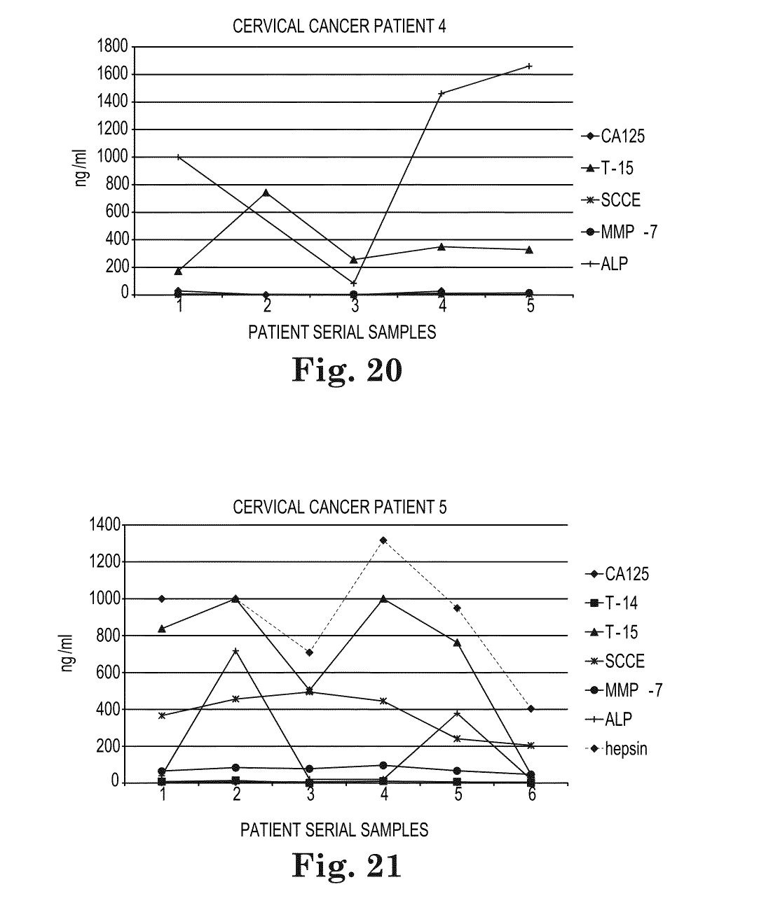

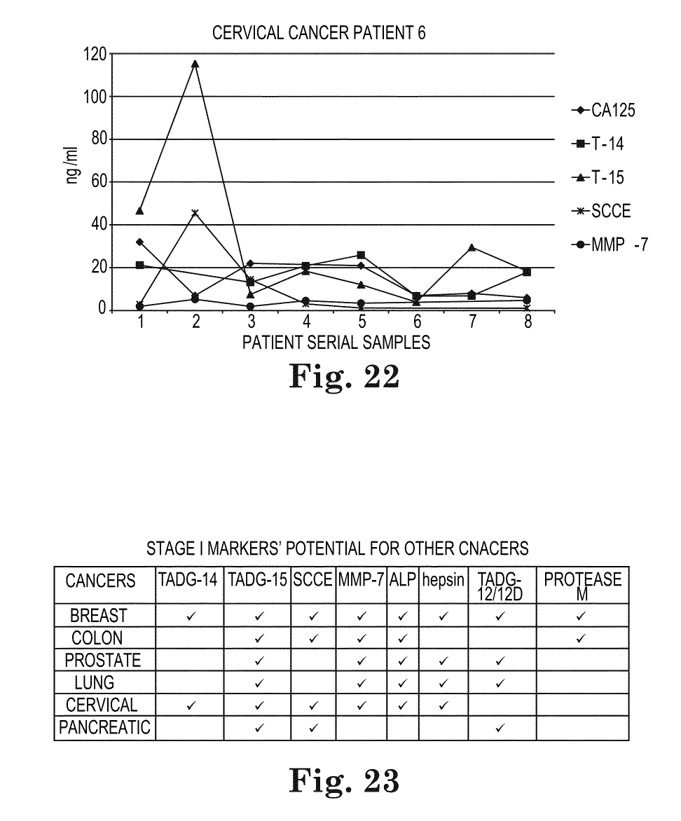

Understanding which proteases are elevated and at which time in tumor progression can be of significant value to caregivers in determining which inhibitory agents may be of value in controlling ongoing tumor growth and spread. Furthermore, it may be pertinent which enzymes are sentinel to the disease process and allow selection of these enzymes for inhibition with the resultant downstream mitigation of a protease cascade and consequently tumor growth and spread. Importantly, because these enzymes are potentially constitutive drivers of neoplasia it could well be that such activities are central to all tumor growth and spread and as such monitoring of patients with other cancers may provide intervention opportunities for reduction in tumor growth and spread via protease inhibition. An example of the expression of these proteases in patients with cervical cancer would suggest that the markers may be beneficial in monitoring patients with cancers other than ovarian cancer (FIGS. 17-22).

The fact that there are well established inhibitors for serine proteases including small molecules, peptides, and native protein inhibitors, several of which have already been demonstrated to reduce tumor growth and spread in animal models (references 3 and 5-10) suggests a capacity to develop new therapies based on the presence of specific proteases being detected in the serum of cancer patients.

Finally, we continue to assess the value of these markers as indicators of early diagnosis. This effort is to some extent restricted by the fact that serum from early stage patients is generally limited and as such limits the number of serum candidates available. In our tumor serum bank we have identified 29 patients with Stage I and II disease and we analyzed these patients with individual serum assays to determine if these markers were elevated. This data is shown in Table 3.

For early stage tumors, 24 of 29 were shown to be elevated (i.e., above the 95% level of the normal population). A combination of these markers along with CA125 could detect 87% of early stage disease compared to 28% for CA125 alone (Table 3). Markers were elevated 87% of the time for late stage and sub-categories also on average. It should be noted that not all the marker data is yet available, e.g. Hepsin for many categories and TADG-12D for all categories due to test availability.

TABLE 3
Detection of ovarian cancer by immunoassays for the listed proteins
in serum at levels above the level of 95% of the normal population.

| | | | | | | | Other |
|---|---|---|---|---|---|---|---|
| | | | | | | Clear | (granulosa, |
| | Stage | Stage | Pap. Serous + | Mucinous | Endometrioid | Cell | germ cell, |
| Assay | I&II | III&IV | Serous Ca. | Ca. | Ca. | Ca. | etc.) |
| CA125 | 8 | 39 | 17 | 4 | 4 | 2 | 6 |
| TADG14 | 3 | 12 | 6 | 0 | 3 | 3 | 2 |
| TADG15 | 12 | 24 | 12 | 7 | 3 | 1 | 5 |
| SCCE | 3 | 5 | 3 | 1 | 0 | 1 | 2 |
| MMP-7 | 4 | 6 | 3 | 1 | 1 | 1 | 0 |
| ALP | 11 | 19 | 10 | 0 | 6 | 2 | 2 |
| Hepsin | 6 | ND | ND | ND | ND | ND | ND |
| Total | 24/29 | 62/70 | 31/39 | 7/8 | 13/15 | 7/9 | 9/9 |
| % | 87% | 89% | 80% | 88% | 87% | 78% | 100% |

ND = No Data Available at this time

Table 3 demonstrates the potential of using this combination of markers for early detection of ovarian cancer. In serum of stage I & II ovarian cancer patients, at least one of the markers CA125, TADG14, TADG15, SCCE, MMP-7, ALP, and hepsin, was elevated above the 95% cut off for the normal range in 24 of 29 patients. CA125, TADG15, and ALP were particularly likely to be elevated. Table 4 shows that 62% (18 of 29) of Stage I & II ovarian cancer patients had serum elevated in either CA125 or TADG15. Table 5 shows that adding ALP to the combination of CA125 and TADG15 would allow identification of 79% (23 of 29) of Stage I & II ovarian cancer patients.

Table 6 shows updated data showing that at least one of the proteins of CA125, TADG14, TADG15, SCCE, MMP-7, hepsin, and ALP, was elevated in 86% of early stage ovarian cancer patients. Thus, this panel of markers, and especially the group consisting of CA125, TADG15, and ALP, allows identification of the large majority of early stage ovarian cancer patients.

TABLE 4
CA125 and TADG-15 expression in Stage I&II, Stage III&IV, and
sub-types of ovarian cancer, by serum immunassays for the proteins.
95% of normal values used as cut-off for over-expression.

| | | | | | | | Other |
|---|---|---|---|---|---|---|---|
| | | | | | | Clear | (Granulosa, |
| | Stage | Stage | Pap. Ser. & | Mucinous | Endometrioid | Cell | Germ Cell, |
| Assay | I&II | III&IV | Serous Ca. | Ca. | Ca. | Ca. | etc.) |
| CA125 | 8/27 | 39/61 | 17/35 | 4/7 | 4/12 | 2/6 | 6/7 |
| TADG-15 | 12/28 | 24/67 | 12/38 | 6/8 | 3/14 | 1/9 | 5/9 |
| Total | 18/29 | 51/69 | 26/39 | 7/8 | 6/15 | 3/9 | 9/9 |
| % | 62% | 74% | 67% | 88% | 40% | 33% | 100% |

TABLE 5
CA125, TADG-15, and ALP expression in Stage I&II, Stage III&IV,
and sub-types of ovarian cancer by serum immunoassays for the proteins.
95% of normal values used as cut-off for over-expression.

| | | | | | | | Other |
|---|---|---|---|---|---|---|---|
| | | | | | | Clear | (Granulosa, |
| | Stage | Stage | Pap. Ser. & | Mucinous | Endometrioid | Cell | Germ Cell, |
| Assay | I&II | III&IV | Serous Ca. | Ca. | Ca. | Ca. | etc.) |
| CA125 | 8/27 | 39/61 | 17/35 | 4/7 | 4/12 | 2/6 | 6/7 |
| TADG-15 | 12/28 | 24/67 | 12/38 | 6/8 | 3/14 | 1/9 | 5/9 |
| ALP | 11/26 | 18/58 | 9/33 | 0/5 | 6/14 | 2/8 | 2/5 |
| Total | 23/29 | 58/70 | 30/39 | 7/8 | 12/15 | 5/9 | 9/9 |
| % | 79% | 83% | 77% | 88% | 80% | 56% | 100% |

TABLE 6
CA125 plus all markers expression in Stage I&II, Stage III&IV,
and sub-types of ovarian cancer by serum immunassays for the proteins.
95% of normal values used as cutoff for over-expression.

| | | | | | | | Other |
|---|---|---|---|---|---|---|---|
| | | | | | | Clear | (Granulosa, |
| | Stage | Stage | Pap. Ser. & | Mucinous | Endometrioid | Cell | Germ Cell, |
| Assay | I&II | III&IV | Serous Ca. | Ca. | Ca. | Ca. | etc.) |
| CA125 | 8/27 | 39/61 | 17/35 | 4/7 | 4/12 | 2/6 | 6/7 |
| TADG-14 | 3/28 | 12/67 | 6/37 | 0/6 | 3/15 | 3/9 | 2/9 |
| TADG-15 | 12/28 | 23/67 | 12/38 | 6/8 | 3/14 | 1/9 | 5/9 |
| SCCE | 3/19 | 6/48 | 3/27 | 1/5 | 0/8 | 1/5 | 2/6 |
| MMP-7 | 4/25 | 6/63 | 3/36 | 1/6 | 1/13 | 1/7 | 0/8 |
| Hepsin | 6/19 | ND | ND | ND | ND | ND | ND |
| ALP | 11/26 | 18/58 | 9/33 | 0/5 | 6/14 | 2/8 | 2/5 |
| Total | 25/29 | 62/70 | 31/39 | 7/8 | 13/15 | 7/9 | 9/9 |
| % | 86% | 89% | 79% | 88% | 87% | 78% | 100% |

ND = No data available at this time.

Most likely, if these markers are used to screen the general population for early diagnosis, an elevated level of the markers should be followed up with imaging studies, biopsy, or other tests as appropriate to confirm a diagnosis of cancer.

These markers are not limited to ovarian cancer. FIGS. 17-20 show that several of the markers are elevated in cervical cancer patients as well. And the applicants believe the markers will be elevated in many other types of cancer. CA125 for instance, is known to be frequently elevated in lymphoma. Table 4 is summary of cancers for which the markers are elevated.

The proteases in this group—TADG12, TADG14, TADG15, SCCE, MMP-7, and hepsin—are potential therapeutic targets. There exist several inhibitors of these proteases that can be administered to patients orally or in some cases by subcutaneous, intraperitoneal, or intravenous injection. The inhibitors of these proteases include the ALP protein in our panel, as well as Bowman-Birk inhibitor, aprotinin, HAI-1, PEBP (phosphatidylethonolamine-binding protein), and FOY-305 (FOYPAN). Antibodies against the enzymes, especially antibodies against the active sites of these enzymes, are also inhibitors of the proteases. Peptides that are inhibitors of the protease or proteases, and other protease inhibitors, are usually quite resistant to digestion, and therefore can be given orally and will be taken into the bloodstream in good yield without degradation.

The term “inhibitor” of a protease as used herein refers to a compound that decreases protease activity in vivo. The inhibitor can act by reducing expression level of the protease (e.g., an antisense RNA), by otherwise reducing the amount of protease in serum or the half-life of the protease (e.g., an antibody targets the protease for destruction by the immune system), or by binding to the enzyme and thereby altering its enzyme activity (i.e., as a competitive, noncompetitive, or uncompetitve enzyme inhibitor). The third category are “kinetic inhibitors.” The term “kinetic inhibitor” as used herein refers to a substance that reduces the activity of the protease in an in vitro assay—that, decreases the k_{cat}/K_{M} of the protease in an in vitro assay, where k_{cat} is the catalytic constant and K_{M} is the Michaelis-Menten constant of the protease enzyme.

An antibody against a protease would be expected to be an inhibitor of the protease at least by removing it from circulation in vivo. The antibody may or may not also be a kinetic inhibitor of the protease. For instance, if the antibody binds to the protease active site it would probably be a kinetic inhibitor that reduces the protease's activity in an in vitro assay.

Monoclonal or polyclonal antibodies produced as described in the examples above can be screened as inhibitors of the protease in vivo and can be screened as kinetic inhibitors by in vitro assays.

Thus, new inhibitors can be found by making monoclonal antibodies against the protease and screening the monoclonal antibodies for inhibiting enzyme activity of the protease in vitro.

Peptide inhibitors can also be found by screening a phage display library of random peptides, for instance random 9-mer peptides, for binding to the protease in question. Detection of protease binding can be done by performing the binding with biotin-labeled protease, and then detecting the biotin by a streptavidin-coupled horse radish peroxidase assay. Or monoclonal or polyclonal antibodies against the protease can be used to detect the protease after it has been contacted with a phage display library. (Detecting for instance, a mouse antibody against the protease with a goat anti-mouse antibody coupled to an enzyme detection system.

Small molecule libraries can also be screened in a protease assay for inhibition of a particular protease to identify new small molecule inhibitors of a particular protease.

Thus, one embodiment of the invention provides a method of identifying an anti-cancer agent comprising: (1) identifying an inhibitor of a protease selected from the group consisting of TADG12, TADG14, TADG15, SCCE, MMP-7, and hepsin (e.g., by one of the methods described above); and (2) testing the inhibitor for anti-cancer activity in an in vivo system against a tumor overexpressing the protease. The in vivo system may be, for instance, a human clinical trial or a mouse allograft or xenograft system.

One embodiment of the invention provides a method of identifying an inhibitor of a protease selected from the group consisting of TADG12, TADG14, TADG15, SCCE, MMP-7, and hepsin. In one embodiment, the method comprises generating an antibody against the protease, and testing the antibody for inhibiting the protease in an in vitro protease enzyme assay. In another embodiment, the method comprises testing a small molecule candidate inhibitor for inhibiting the protease in an in vitro protease enzyme assay. In another embodiment, the method comprises testing the protease for binding to a phage display peptide library, identifying a peptide from the library bound by the protease, and testing the peptide for inhibition of the protease in an in vitro protease enzyme assay.

It is interesting how the pattern of expression of the proteases shifts during cancer treatment, as seen in the figures discussed above. One protease may be expressed at high levels and then dip in its level and be replaced by another protease or combination of proteases. And then later in the same patient, the first protease may become elevated again. So the particular proteases being expressed by a tumor shifts over time. Thus, it is advantageous to monitor the protease levels with treatment, and select inhibitors for therapeutic administration that inhibit the proteases currently being expressed.

The inhibitors used may shift during treatment as different proteases become elevated or decrease in level. Thus, it may be observed that protease 1 is elevated in a patient initially and an inhibitor for protease 1 is administered. After a time, a new serum assay of the patient may show that protease 1 has decreased in level and protease 2 is now elevated. At that time, the administration of the inhibitor of protease 1 may be discontinued and an inhibitor for protease 2 given.

Many protease inhibitors are known. These include FOY-305 (15, 4, 5, 6), FO-349 (18), ONO-3403 (4, 9), FOY-251 (15), heparin (5), serpins including antithrombin III (13), ecotin and ecotin M84R/M85R (16, 17), CU-697, CU-698, and several other small molecules disclosed in (8).

Protease inhibitors have varying selectivity for different proteases. In vitro assays are known and disclosed in many of the cited references, including (8, 15), and can be used to determine the half maximal inhibitory concentration (IC₅₀) of a particular inhibitor for a particular protease. In this way, which inhibitors are selective or effective for which proteases can be determined.

Most protease inhibitors are resistant to digestion and therefore can be given orally. An effective dose for FOY-305 orally in mice is as 0.1% of the food, and in humans at a range of approximately 100 mg to 1 g per day. An effective dose by intraperitoneal injection in mice is 20 mg/kg (5). The IC₅₀ of FOY-305 for plasma kallikrein is 1.5 uM (15). Effective doses of other protease inhibitors in vivo can be estimated by comparing their IC₅₀s for targeted proteases to that of FOY-305.

The protease inhibitors may be given to humans or laboratory animals to treat cancer orally, or by intravenous, subcutaneous, or intraperitoneal injection.

Methods of treating cancer in vivo with protease inhibitors are described in references 3 and 5-10 below.

Thus, one embodiment of the invention provides a method of early diagnosis of cancer comprising: (a) measuring in a human blood sample protein levels of two or more proteins selected from the group consisting of CA125, TADG14, TADG15, TADG12, SCCE, MMP-7, ALP, KLK6, and hepsin; and (b) comparing the levels of the two or more proteins to normal range levels of the two or more proteins to identify whether the level of at least one of the two or more proteins is elevated. If at least one of the levels of the two or more proteins is elevated, the human may have cancer.

The method typically further involves conducting one or more further tests on the human to identify if the human has cancer.

The blood sample may be serum, or whole blood, or plasma, or other fractionated blood product. In specific embodiments, the further test comprise an imaging method, for instance an x-ray, a computerized axial tomography scan (CT scan), a magnetic resonance imaging (MRI), a positron emission tomography scan (PET scan), or an ultrasound.

In specific embodiments, the further tests comprise a biopsy.

In a specific embodiment, the cancer is ovarian cancer.

In another specific embodiment, the cancer is cervical cancer.

In other specific embodiments, the cancer is prostate cancer, breast cancer, pancreatic cancer, or kidney cancer.

In a more specific embodiment, the method comprises comparing the levels of the two or more proteins to normal range levels of the two or more proteins to identify whether the levels of the at least two of the two or more proteins are elevated; and if at least two of the two or more proteins are elevated, conducting one or more further tests on the human to identify if the human has cancer.

In one embodiment, the method comprises: (a) measuring in a human serum protein levels of two or more proteins selected from the group consisting of CA125, TADG15, and ALP (or all three proteins); and (b) comparing the levels of the two or more proteins (or all three proteins) to normal range levels of the two or more proteins (or all three proteins) to identify whether the level of at least one of the two or more proteins (or at least one of the three proteins) is elevated.

In a specific embodiment, the two or more proteins are selected from the group consisting of TADG14, TADG15, TADG12, SCCE, MMP-7, ALP, and hepsin.

In another specific embodiment, the two or more proteins are selected from the group consisting of CA125, TADG14, TADG15, TADG12, SCCE, MMP-7, ALP, and hepsin;

In another specific embodiment, the two or more proteins are selected from the group consisting of CA125, TADG15, and hepsin.

In another embodiment, the method comprises: (a) measuring in a human serum protein levels of CA125, TADG15, and hepsin; and (b) comparing the levels of the three proteins to normal range levels of the three proteins to identify whether the level of at least one of the three is elevated.

In another embodiment, the method of early diagnosis of cancer comprises: (a) measuring in the cancer patient serum protein levels of three or more (or 4 or more, 5 or more, 6 or more, 7 or more, or all) of the proteins selected from the group consisting of CA125, TADG14, TADG15, TADG12, SCCE, MMP-7, ALP, KLK6, and hepsin; and (b) comparing the levels of the three or more (or 4 or more, 5 or more, 6 or more, 7 or more, or all of the) proteins to normal range levels of the three or more (or 4 or more, 5 or more, 6 or more, 7 or more, or all of the) proteins to identify whether the level of at least one (or at least 2 or at least 3) of the three or more (or 4 or more, 5 or more, 6 or more, 7 or more, or all of the) proteins is elevated above normal range levels or is increasing or decreasing.

Another embodiment provides a method of early diagnosis of cancer comprising: (a) measuring in a human blood sample protein levels of two or more proteins selected from the group consisting of CA125, TADG14, TADG15, TADG12, SCCE, MMP-7, ALP, KLK6, and hepsin; (b) comparing the levels of the two or more proteins to normal range levels of the two or more proteins to identify whether the level of at least two of the two or more proteins is elevated, the human may have cancer. In a specific embodiment. In a specific embodiment, if at least two of the two or more proteins is elevated, the method comprises diagnosing the human as having cancer. In a specific embodiment, the cancer is ovarian cancer.

Another embodiment provides a method of monitoring progress of cancer in a cancer patient comprising: (a) measuring in the cancer patient blood protein levels of two or more proteins selected from the group consisting of CA125, TADG14, TADG15, TADG12, SCCE, MMP-7, ALP, KLK6, and hepsin; and (b) comparing the levels of the two or more proteins to normal range levels of the two or more proteins and/or to previous levels of the two or more proteins in the same patient to identify whether the level of at least one of the two or more proteins is elevated above normal range levels or is increasing or decreasing.

In a more specific embodiment, the two or more proteins are selected from the group consisting of CA125, TADG14, TADG15, TADG12, SCCE, MMP-7, ALP, and hepsin.

In a more specific embodiment, the method comprises (a) measuring in the cancer patient blood protein levels of one or more proteases selected from the group consisting of TADG12, TADG14, TADG15, SCCE, MMP-7, KLK6, and hepsin; comparing the levels of the one or more proteases to normal range levels of the one or more proteases and/or to previous levels of the one or more proteases in the same patient to identify whether the level of at least one of the one or more proteases is elevated above normal range levels or is increasing or decreasing; and if the level of at least one of the one or more proteases is elevated or increasing, treating the patient with an inhibitor of the one or more proteases whose level is elevated or increasing.

In specific embodiments, the protease inhibitor may be Bowman-Birk inhibitor, ALP, aprotinin, HAI-1, PEBP (phosphatidylethanoloamine-binding protein), FOY-305 (FOYPAN), probucol, or an antibody or antibodies against one or more of the one or more proteases whose level is elevated or increasing.

In a specific embodiment of the method of monitoring progress of cancer in a cancer patient (with or without treating with a protease inhibitor) the cancer is ovarian cancer. In other specific embodiments, the cancer is a cancer other than ovarian cancer, for instance cervical cancer. In other specific embodiments, the cancer is prostate cancer, breast cancer, pancreatic cancer, or kidney cancer.

In a specific embodiment of monitoring progress of cancer in a cancer patient, the method comprises (a) measuring in the cancer patient blood protein levels of three or more (or 4 or more, 5 or more, 6 or more, 7 or more, or all) of the proteins selected from the group consisting of CA125, TADG14, TADG15, TADG12, SCCE, MMP-7, ALP, KLK6, and hepsin; and (b) comparing the levels of the three or more (or 4 or more, 5 or more, 6 or more, 7 or more, or all of the) proteins to normal range levels of the three or more (or 4 or more, 5 or more, 6 or more, 7 or more, or all of the) proteins and/or to previous levels of the three or more (or 4 or more, 5 or more, 6 or more, 7 or more, or all of the) proteins in the same patient to identify whether the level of at least one of the three or more (or 4 or more, 5 or more, 6 or more, 7 or more, or all of the) proteins is elevated above normal range levels or is increasing or decreasing.

In a specific embodiment, the method comprises measuring in the cancer patient blood protein levels of the proteins CA125, TADG15, and ALP (or two or more proteins from the group consisting of CA125, TADG15, and ALP); and comparing the levels of the proteins to normal range levels of the proteins and/or to previous levels of the proteins in the same patient to identify whether the level of at least one of the proteins is elevated above normal range levels or is increasing or decreasing.

In a specific embodiment, the method comprises measuring in the cancer patient blood protein levels of the proteins CA125, TADG15, and hepsin (or two or more proteins from the group consisting of CA125, TADG15, and hepsin); and comparing the levels of the proteins to normal range levels of the proteins and/or to previous levels of the proteins in the same patient to identify whether the level of at least one of the proteins is elevated above normal range levels or is increasing or decreasing.

The method of early diagnosis of cancer or of monitoring cancer can also be used to establish a prognosis for the cancer patient. The levels of one or a combination of the proteins listed can over time be linked to differential outcomes for cancer patients, possibly depending on the treatment chosen.

Another embodiment provides a method of treating cancer comprising administering a protease inhibitor.

In a specific embodiment, the protease inhibitor is Bowman-Birk inhibitor, ALP, aprotinin, HAI-1, PEBP (phosphatidylethanoloamine-binding protein), FOY-305 (FOYPAN), probucol, or an antibody or antibodies against one or more of the proteases selected from the group consisting of: TADG12, TADG14, TADG15, SCCE, MMP-7, KLK6, and hepsin.

In a specific embodiment, the antibody or antibodies are against one or more proteases are selected from the group consisting of TADG12, TADG14, TADG15, SCCE, MMP-7, and hepsin.

In one embodiment, the protease inhibitor a cyclic peptide of the formula I:

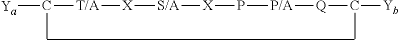

where Yₐ and Y_{b} are each optionally present, and if present are a peptide of 1-11 amino acid residues, and where Yₐ and Y_{b} collectively comprise 0-11 amino acid residues.

Another embodiment provides a method of treating cancer comprising: measuring in a cancer patient blood (e.g., whole blood, serum, or plasma) protein levels of one or more proteases selected from the group consisting of TADG12, TADG14, TADG15, SCCE, MMP-7, KLK6, and hepsin; comparing the levels of the one or more proteases to normal range levels of the one or more proteases and/or to previous levels of the one or more proteases in the same patient to identify whether the level of at least one of the one or more proteases is elevated above normal range levels or is increasing; and if the level of at least one of the one or more proteases is elevated or increasing, treating the patient with an inhibitor of the elevated or increasing at least one protease.

In a more specific embodiment, the one or more proteases are selected from the group consisting of TADG12, TADG14, TADG15, SCCE, MMP-7, KLK6, and hepsin.

In another specific embodiment, the protease is selected from the group consisting of TADG12, TADG14, TADG15, SCCE, MMP-7, and hepsin.

In one embodiment wherein the level of a first protease of the one or more proteases is elevated or increasing and the method comprises treating the patient with a first inhibitor that inhibits the first protease; the method may further comprise: repeating the measuring and comparing steps at least one week after the treatment; and if the level of a second protease of the one or more proteases is elevated or increasing, treating the patient with a second inhibitor that inhibits the second protease, wherein the first inhibitor and the second inhibitor are different inhibitors and the first protease and second protease are different proteases.

In a more specific embodiment, the second inhibitor does not inhibit the first protease.

In a specific embodiment of the method of treating a patient with a protease inhibitor, the inhibitor is a kinetic inhibitor of the elevated or increasing at least one protease.

In another embodiment, the inhibitor is an antibody against the elevated or increasing at least one protease. The antibody may be a kinetic inhibitor as well.

### BIBLIOGRAPHY

1. Hansson, L., Stromqvist, M., Backman, A., Wallbrandt, P., Carlstein, A., Egelrud, T., Cloning, Expression, and Characterization of Stratum Corneum Chymotryptic Enzyme. A Skin-Specific Human Serine Protease, Journal of Biological Chemistry, 269(30): 19420-19426, Jul. 29 (1994)
2. Liu, Y., Identifying Breast Cancer Targets for Protease Inhibitors, Scripps Research Institute Grant #6FB-0066 (2000)
3. Ohkoshi, M., Sasaki, Y., Antimetastatic Activity of a Synthetic Serine Protease Inhibitor, FOY-305 (Foypan®), In Vivo, January-February; 19(1):133-136 (2005)
4. Ohkoshi, M., Denda, T., Hiwasa, T. Growth-Suppressive Activities of Serine Protease Inhibitors, FOY-305, ONO-3403 and FO-349 Toward Human Carcinoma Cells, Oncology Reports, Vol. 4, Num. 3: 521-523, May (1997)
5. Ohkoshi, M., Akagawa, T., Nakajima, M., Effects of Serine Protease Inhibitor FOY-305 and Heparin on the Growth of Squamous Cell Carcinoma, Anticancer Res. July-August: 13(4): 963-966 (1993)
6. Ohta, T., Futagami, F., Tajima, H., Kitagawa, H., Kayahara, M., Nagakawa, T., Miwa, K., Yamamoto, M., Iseki, S., Nakanuma, Y., Terada, T., Inhibitory Effect of a Serine Protease Inhibitor, FOY-305 on the Invasion and Metastasis of Human Pancreatic Cancers, International Journal of Oncology, Vol. 11, Num. 4: 813-817, October (1997)
7. Takeuchi, T., Shuman, M A., Craik, C S., Reverse Biochemistry: Use of Macromolecular Protease Inhibitors to Dissect Complex Biological Processes and Identify a Membrane-Type Serine Protease in Epithelial Cancer and Normal Tissue, Proc. Natl. Acad. Sci., Sep. 28: 96(20): 11054-11061 (1999)
8. Uhland, K., Siphos, B., Arkona, C., Schuster, M., Petri, B., Steinmetzer, P., Mueller, F., Schweinitz, A., Steinmetzer, T., Van de Locht, A., Use of IHC and Newly Designed Matriptase Inhibitors to Elucidate the Role of Matriptase in Pancreatic Ductal Adenocarcinoma, International Journal of Oncology, 35: 347-357 (2009)
9. Engi H, et al., Modelling of tomour—Host coexistence in vitro in the presence of serine protease inhibitors. In Vivo. 23(5): 711-715. 2009.
10. Lee M S, Matrix-degrading type II transmembrane serine protease Matriptase: Its role in cancer development and malignancy. Journal of Cancer Molecules. 2(5): 183-190, 2006.
11. Chevillet J R, et al., Identification and characterization of small-molecule inhibitors of hepsin. Mol. Cancer Ther. 7(10): 3343-3351, 2008.
12. White, N M A et al. KLK6 and KLK13 predict tumor recurrence in epithelial ovarian carcinoma. Br. J. Cancer (2009) 101:1107-1113.
13. Magklara, A. et al. Characterization of the enzymatic activity of human kalikrein 6: autoactivation, substrate specificity, and regulation by inhibitors, Biochem. Biophys. Res. Comm. (2003) 307:948-955.
14. McBride et al. Synthetic peptide mimics of the Bowman-Birk inhibitor protein, Curr. Med. Chem. 2001, 8:909-917.
15. Senokuchi, K. et al. 1995. New orally active serine protease inhibitors. J. Med. Chem. 38:2521-2523.
16. Yang S Q, Wang C I, Gillmor S A, Fletterick R J, Craik C S. Ecotin: a serine protease inhibitor with two distinct and interacting binding sites. J Mol Biol. 1998 Jun. 19; 279(4):945-57.
17. Wang C I, Yang Q, Craik C S. Isolation of a high affinity inhibitor of urokinase-type plasminogen activator by phage display of ecotin. J Biol Chem. 1995 May 19; 270(20):12250-6.
18. Ikeda, T. et al. 1998. Anti-invasive activity of synthetic serine protease inhibitors and its combined effect with a matrix metalloproteinase inhibitor. Anticancer Res. 18(6A): 4259-65.

All references cited are incorporated by reference.

What is claimed is:

1. A method of early diagnosis of cancer comprising:
measuring in a human blood sample protein levels of two or more proteins selected from the group consisting of CA125, TADG14, TADG15, TADG12, SCCE, MMP-7, ALP, KLK6, and hepsin; and
comparing the levels of the two or more proteins to normal range levels of the two or more proteins to identify whether the level of at least one of the two or more proteins is elevated.

2. The method of claim 1 wherein the method further comprises if at least one of the levels of the two or more proteins is elevated conducting one or more further tests on the human to identify if the human has cancer.

3. The method of claim 2 wherein the further tests comprise an imaging method selected from the group consisting of an X-ray, a CT scan, an MRI, a PET scan, and an ultrasound.

4. The method of claim 2 wherein the further tests comprise a biopsy.

5. The method of claim 1 wherein the cancer is ovarian cancer.

6. The method of claim 1 wherein the method comprises:
comparing the levels of the two or more proteins to normal range levels of the two or more proteins to identify whether the levels of at least two of the two or more proteins are elevated.

7. The method of claim 6 wherein the method further comprises, if at least two of the levels of the two or more proteins are elevated, conducting one or more further tests on the human to identify if the human has cancer.

8. The method of claim 1 wherein the method comprises:
measuring in a human serum protein levels of two or more proteins selected from the group consisting of CA125, TADG15, and ALP; and
comparing the levels of the two or more proteins to normal range levels of the two or more proteins to identify whether the level of at least one of the two or more proteins is elevated.

9. The method of claim 8 wherein the method further comprises, if at least one of the levels of the two or more proteins is elevated, conducting one or more further tests on the human to identify if the human has cancer.

10. The method of claim 1 wherein the two or more proteins are selected from the group consisting of TADG14, TADG15, TADG12, SCCE, MMP-7, ALP, and hepsin.

11. The method of claim 1 wherein the two or more proteins are selected from the group consisting of CA125, TADG14, TADG15, TADG12, SCCE, MMP-7, ALP, and hepsin;

12. The method of claim 1 wherein the two or more proteins are selected from the group consisting of CA125, TADG15, and hepsin.

13. The method of claim 1 comprising:
measuring in the cancer patient serum protein levels of three or more of the proteins selected from the group consisting of CA125, TADG14, TADG15, TADG12, SCCE, MMP-7, ALP, KLK6, and hepsin; and
comparing the levels of the three or more proteins to normal range levels of the three or more proteins to identify whether the level of at least one of the three or more proteins is elevated above normal range levels or is increasing or decreasing.

14. The method of claim 13 wherein the three or more proteins are selected from the group consisting of CA125, TADG14, TADG15, TADG12, SCCE, MMP-7, ALP, and hepsin.

15. A method of monitoring progress of cancer in a cancer patient comprising:
measuring in the cancer patient blood protein levels of two or more proteins selected from the group consisting of CA125, TADG14, TADG15, TADG12, SCCE, MMP-7, ALP, KLK6, and hepsin; and
comparing the levels of the two or more proteins to normal range levels of the two or more proteins and/or to previous levels of the two or more proteins in the same patient to identify whether the level of at least one of the two or more proteins is elevated above normal range levels or is increasing or decreasing.

16. The method of claim 15 wherein the two or more proteins are selected from the group consisting of CA125, TADG14, TADG15, TADG12, SCCE, MMP-7, ALP, and hepsin.

17. The method of claim 15 comprising:
measuring in the cancer patient blood protein levels of one or more proteases selected from the group consisting of TADG12, TADG14, TADG15, SCCE, MMP-7, KLK6, and hepsin;
comparing the levels of the one or more proteases to normal range levels of the one or more proteases and/or to previous levels of the one or more proteases in the same patient to identify whether the level of at least one of the one or more proteases is elevated above normal range levels or is increasing or decreasing; and
if the level of at least one of the one or more proteases is elevated or increasing, treating the patient with an inhibitor of the one or more proteases whose level is elevated or increasing.

18. The method of claim 17 wherein the protease inhibitor is Bowman-Birk inhibitor, ALP, aprotinin, HAI-1, PEBP (phosphatidylethanoloamine-binding protein), FOY-305 (FOYPAN), probucol, or an antibody or antibodies against one or more of the one or more proteases whose level is elevated or increasing.

19. The method of claim 15 wherein the cancer is ovarian cancer.

20. The method of claim 15 wherein the cancer is a cancer other than ovarian cancer.

21. The method of claim 20 wherein the cancer is cervical cancer.

22. The method of claim 15 comprising:
measuring in the cancer patient blood protein levels of three or more of the proteins selected from the group consisting of CA125, TADG14, TADG15, TADG12, SCCE, MMP-7, ALP, KLK6, and hepsin; and
comparing the levels of the three or more proteins to normal range levels of the three or more proteins and/or to previous levels of the three or more proteins in the same patient to identify whether the level of at least one of the three or more proteins is elevated above normal range levels or is increasing or decreasing.

23. The method of claim 15 wherein the method comprises
measuring in the cancer patient blood protein levels of the proteins CA125, TADG15, and ALP; and
comparing the levels of the proteins to normal range levels of the proteins and/or to previous levels of the proteins in the same patient to identify whether the level of at least one of the proteins is elevated above normal range levels or is increasing or decreasing.

24. The method of claim 15 wherein the method comprises:
measuring in the cancer patient blood protein levels of two or more proteins selected from the group consisting of CA125, TADG15, and ALP; and
comparing the levels of the two or more proteins to normal range levels of the two or more proteins and/or to previous levels of the two or more proteins in the same patient to identify whether the level of at least one of the two or more proteins is elevated above normal range levels or is increasing or decreasing.

25. A method of treating cancer comprising:
measuring in a cancer patient blood protein levels of one or more proteases;
comparing the levels of the one or more proteases to normal range levels of the one or more proteases and/or to previous levels of the one or more proteases in the same patient to identify whether the level of at least one of the one or more proteases is elevated above normal range levels or is increasing; and
if the level of at least one of the one or more proteases is elevated or increasing, treating the patient with an inhibitor of the elevated or increasing at least one protease.

26. The method of claim 25 wherein the one or more proteases are selected from the group consisting of TADG12, TADG14, TADG15, SCCE, MMP-7, KLK6, and hepsin.

27. The method of claim 25 wherein the protease is selected from the group consisting of TADG12, TADG14, TADG15, SCCE, MMP-7, and hepsin.

28. The method of claim 25 wherein the level of a first protease of the one or more proteases is elevated or increasing and the method comprises treating the patient with a first inhibitor that inhibits the first protease; the method further comprising:
repeating the measuring and comparing steps at least one week after the treatment; and
if the level of a second protease of the one or more proteases is elevated or increasing, treating the patient with a second inhibitor that inhibits the second protease, wherein the first inhibitor and the second inhibitor are different inhibitors and the first protease and second protease are different proteases.

29. The method of claim 28 wherein the second inhibitor does not inhibit the first protease.

30. The method of claim 25 wherein the inhibitor is a kinetic inhibitor of the elevated or increasing at least one protease.

31. The method of claim 25 wherein the inhibitor is an antibody against the elevated or increasing at least one protease.

32. The method of claim 31 wherein the antibody is a kinetic inhibitor of the elevated or increasing at least one protease.


SEQUENCE LISTING (5 sequences)

SEQ ID NO: 1 - protein, 9 aa, Artificial
  Protease inhibitor peptide
CTKSNPPQC

SEQ ID NO: 2 - protein, 9 aa, Artificial
  Protease inhibitor peptide
CALSYPAQC

SEQ ID NO: 3 - protein, 234 aa, Homo sapiens
NMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNEMIQPVCLPNSEENFPDGKVCWTSGWGATEDGAGDASPVLNHAAVPLISNKICNHRDVYGGIISPSMLCAGYLTGGVDSCQGDSGGPLVCQERRLWKLVGATSFGIGCAEVNKPGVYTRVTSFLDWIHEQMERDLKT

SEQ ID NO: 4 - protein, 124 aa, Homo sapiens
NMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNGTSGSLCGSAALPLFQEDLQLLIEAFL

SEQ ID NO: 5 - protein, 73 aa, Homo sapiens
NMSLLSQWPWQASLQFQGYHLCGGSVITPLWIITAAHCVYEIVAPRERADRRGRKLLCWRKPTKMKGPRPSHS